(19) United States
(12) Patent Application Publication
(10) Pub. No.: US 2024/0180751 A1
(43) Pub. Date: Jun. 6, 2024

(54) WOUND CARE PRODUCT WITH INTEGRATED SENSOR TECHNOLOGY FOR DETERMINING WOUND DATA

(71) Applicant: KOB GMBH, Wolfstein (DE)

(72) Inventors: Marcin MEYER, Wolfstein (DE); Johanna Katharina PLENKMANN, Moers (DE); Sina BORCZYK, Kaiserslautern (DE); Kai SCHROVENWEVER, Koblenz (DE)

(21) Appl. No.: 18/553,519

(22) PCT Filed: Dec. 30, 2021
(86) PCT No.: PCT/EP2021/087839

§ 371 Date: Sep. 29, 2023

(30) Foreign Application Priority Data
Mar. 30, 2021 (DE) 10 2021 108 084.3

(51) Int. Cl. A61F 13/00

(52) U.S. Cl. CPC A61F 13/00051; A61F 2013/00604; A61F 2013/00944; A61F 2013/00948; A61F 2013/00953

(57) ABSTRACT

The invention relates to a wound dressing in which sensor technology can be integrated for detecting wound data such as pH value, moisture and temperature as well as data relating to the condition of the wound dressing itself.

The invention relates to a wound dressing in which sensors for recording wound data such as pH, moisture, and temperature and also data on the condition of the wound dressing itself can be integrated. The wound dressing comprises a cover layer, at least one foam-containing layer, a wound-contact layer, and at least one textile-based fabric containing at least one sensor. Such a wound dressing can be used for chronic wounds, for example. With this dressing it is possible, by means of the at least one sensor, to continuously collect information in the form of sensor data both on the condition of the wound and on the condition of the wound dressing itself.

The healing of skin wounds is based on the ability of the skin to regenerate epithelial and also connective and supporting tissues. Regeneration itself is characterized by a complex process of interconnecting cell activities that gradually drive the healing process forward. Thus, the literature describes three fundamental phases of wound healing irrespective of wound type. These include the inflammatory or exudative phase for hemostasis and wound cleansing (phase 1, cleansing phase), the proliferative phase for the formation of granulation tissue (phase 2, granulation phase), and the differentiation phase for epithelization and cicatrization (phase 3, epithelization phase).

A wound is understood as meaning a parting of the tissues that envelope the body in humans or animals. It may be associated with a loss of substance.

Chronic wounds are a particularly difficult type of wound to treat and are characterized inter alia by showing only very little or no healing even after a treatment period of eight to ten weeks. Such wounds can accordingly require months of treatment. The associated dressing changes can thus cause a patient pain time and again over a long period of time. The resulting severe reduction in quality of life means that the physical suffering is often accompanied by mental suffering too, which can cause distress to the patient.

Monitoring the wound beds of chronic wounds and the condition of the wound dressing can be one way of helping improve treatment, of long-term patients in particular, since this increases the safety of treatment and can make it possible to avoid frequent dressing changes. By continuously recording wound parameters, such monitoring can also offer the opportunity to conduct more intensive research into wound healing processes.

Monitoring based on the use as sensors of electronic components having rigid housings can cause such housings to exert point pressure on the wound when pressing down, which can cause the patient pain. However, the use of conventional flexible sensors does not always provide adequate resolutions, with the result that reliable measurement results often cannot be obtained.

WO 2017/195038 A1 describes a wound dressing comprising at least one essentially flexible substrate, said substrate bearing one or more sensors. More particularly, a wound dressing is described in which it is possible to integrate a film-based substrate, on the surface of which is printed/mounted at least one sensor for monitoring wound data. Such a wound dressing appears to have scope for improvement as regards the possibility of absorbency and the application of the sensors.

An object of the present invention is to overcome the disadvantages of the prior art and to further improve the treatment of wounds and of chronic wounds in particular. In particular, the present invention should have an advantageous influence on wound healing in the epithelization or regeneration phase, thereby making it possible for example to shorten the duration of treatment and/or lessen scarring. In addition, the present invention should provide a wound-care product that makes it possible to maximize the efficacy of treatment. Moreover, the treatment should not be perceived as unpleasant by the patient, so that high patient compliance (adherence to the treatment instructions by the patient) is achieved. Pain during dressing changes should be reduced to the absolute minimum. It should also be possible to apply the wound-care product in an advantageous manner.

The objects were unexpectedly achieved by a multilayer (multi-ply) wound-care product having specially integrated sensors for recording wound parameters and the condition of the wound-care product itself. In addition, the structure of the wound-care product of the invention makes it possible for the data/information logged by the sensors to be recorded in a minimally invasive manner.

### The invention accordingly provides a wound dressing comprising:

(a) cover layer,
(b) at least one foam-containing layer,
(c) wound-contact layer, and
(d) at least one textile-based fabric containing at least one sensor.

The novel wound dressing of the invention is distinguished by a number of unexpected advantages.

The presence of at least one sensor in the at least one textile-based fabric means that the at least one sensor is less susceptible to interference from, for example, pressure exerted from the side facing away from the wound. In addition, the arrangement according to the invention of the at least one textile-based fabric containing at least one sensor achieves high accuracy of measurement of the logged data.

The sensors present in the wound dressing make it possible to inspect and monitor the condition both of the wound and of the foam-containing layer at any time with only minimal effort, without the need to remove the wound dressing from the wound. This makes it possible to reduce the number of wound dressing changes to the minimum necessary, which brings an advantageous reduction for the patient in the trauma often associated with such changes. It also boosts patient compliance. Where reference is made herein to monitoring of the “condition of the wound” by means of a sensor, this refers to the recording and, if necessary, evaluation of sensor data, which allow conclusions to be drawn about certain, in particular physically, measurable aspects of the condition of the wound. Sensor-based monitoring of the “condition of the wound” includes for example measurement of the temperature of the wound, since inflamed wounds typically have an elevated temperature. The monitoring/characterization of the condition of the wound can for example utilize also the pH of the wound exudate or the amount of wound exudate secreted by the wound. “Condition of the foam-containing layer” is understood as meaning for example the amount of fluid present in the wound dressing or the pH thereof.

A low frequency of dressing change can also bring savings both in time for the person giving treatment and in resources (raw materials needed for production of a dressing).

The constituents (a) to (d) of the wound dressing of the invention are described below.

The wound dressing of the invention includes a cover layer (a) for closing the wound space. A wound space is understood as meaning the wound and, where appropriate, the adjoining wound environs.

In a preferred embodiment of the invention, the cover layer is a film. The cover layer material is preferably a water-insoluble polymer or a metal. In a preferred embodiment of the invention, the cover layer material is a water-insoluble polymer. The water-insoluble polymer preferably has a solubility in H₂O of 10 mg/l or less, more preferably of 1 mg/ml or less, especially of 0.0001 to 1 mg/ml (determined by the column elution method according to EU Directive 67-548-EEC, Annex V, Chapter A6). Examples include polyurethane, polyether urethane, polyester urethane, polyester, polypropylene, polyethylene, polyamide or polyvinyl chloride, polyorganosiloxane (silicone), polyether-polyamide copolymers, polyacrylate or polymethacrylate, or a mixture thereof.

A suitable cover layer (a) is preferably a film composed of a fluid-impermeable and water vapor-permeable polymer film. Examples are polymer films made of polyester, polyurethane, polyether urethane, polyester urethane, polyether-polyamide copolymers, polyacrylate or polymethacrylate. Particular preference as a cover layer is given to a polyester, polyurethane, polyester urethane or polyether urethane film. The permeability to water vapor of the polymer film is preferably at least 300 g/m²/24 h, especially at least 1000 g/m²/24 h, and most preferably at least 2000 g/m²/24 h up to for example 5000 g/m²/24 h or 10 000 g/m²/24 h (measured upright according to DIN EN 13726).

In an alternative embodiment, the cover layer comprises a textile-based fabric. The textile-based fabrics include, irrespective of production technology, various fabrics such as woven fabrics, knitted fabrics, crocheted fabrics, nonwoven fabrics, and braids.

Woven fabrics can be understood as meaning weaving products. Examples include cloth, velvet, and other fabrics composed of special thread arrangements that are essentially perpendicular to one other. The threads in the longitudinal direction are referred to as warp threads and the threads in the transverse direction are called weft threads. In order for the fabric to achieve sufficient strength, the warp and weft threads must be closely interwoven and therefore have a closed appearance.

Knitted fabrics are also known as knits or knitted goods. Knitted fabrics are knitted materials and are mostly produced by machine from thread systems through the formation of stitches.

A crocheted fabric is a textile mesh produced from yarn(s) by thread looping, the stitches being formed in a row one after the other.

A nonwoven fabric should be understood as meaning a sheet material or structure composed of fibers in a directed or random arrangement that have been consolidated mechanically and/or thermally and/or chemically. Nonwoven fabrics (also called nonwovens) are fundamentally different from woven fabrics, braids, crocheted fabrics and knitted fabrics.

A braid is a product created by interlacing several strands of yarn.

The fibers and/or filaments processed further into textile fabrics in yarn or twist form may be of natural or synthetic origin or comprise mixtures thereof. Examples of fibers of natural origin include silk, viscose, cotton, and wool. Fibers and/or filaments of synthetic origin include synthetic polymers (synthetic fibers) such as polyacrylates, polyamides, polyimides, polyamidimides, polyurethanes, polyesters (especially polyethylene terephthalates and polybutylene terephthalates), polyetheresters, polyethers, polyacrylonitriles, polyalkenes (especially polyethylene and polypropylene), and polytetrafluoroethylene.

The cover layer has a thickness preferably of from 15 to 60 μm, especially 20 to 40 μm, and most preferably from 25 to 30 μm.

The cover layer (a) may be covered with an adhesive layer (k). In particularly preferred embodiments, the cover layer has a moisture-tight, water vapor-permeable, and adhesive edge section. This edge section ensures that the wound dressing can be applied and fixed in the correct place. It also ensures that no fluid can escape between the cover layer and the skin surrounding the wound to be treated. Particular preference is given to adhesives that, in a thin application of from 20 to 35 g/m², together with the film have a permeability to water vapor of from 400 to 10 000 g/m²/24 h, and preferably from 1000 to 5000 g/m²/24 h (measured upright according to DIN EN 13726).

The wound dressing of the invention includes at least one foam-containing layer (b). In a preferred embodiment, the wound dressing of the invention includes a foam-containing layer. In a further preferred embodiment, the wound dressing of the invention includes two foam-containing layers.

Foams are normally understood as meaning materials having cells (open, closed or both) distributed throughout the material. Such materials therefore normally have a bulk density (in accordance with DIN EN ISO 845) lower than the density of the matrix substance. All plastics/polymers capable of being foamed are essentially suitable for producing foams. Examples are polyurethane foams, silicone foams or polystyrene foam.

In a preferred embodiment, the foam is an absorbent foam. An absorbent foam is understood as meaning a foam that can absorb a fluid such as a wound exudate into its polymer matrix and/or its pores and retain (absorb) it there. Preference is given to using open-cell foams as foams. This can prevent for example wound exudate and thus the harmful/toxic substances contained therein from getting back into the wound.

Preference is given to foams having a high absorption capacity. This absorption capacity should be present even when the foam has already absorbed a proportion of its own weight of water into its polymer matrix. Preferably, a foam can have a water content of at least 10% by weight and not more than 80% by weight of water and have a free absorption A₂ of at least 10 g/g, especially at least 12 g/g, and most preferably at least 15 g/g, the free absorption A₂ being determined in accordance with DIN-EN 13726-1 (2002). The free absorption A₂ is here the free absorption of the water-containing foam.

According to a further preferred embodiment of the invention, the foam can have a cell count (=number of pores along a line per linear inch) of at least 5 and not more than 400 cells per inch. The cell count is preferably determined microscopically.

In a preferred embodiment, the foam may have a retention value R of at least 20%, preferably at least 30%, especially at least 40%, and most preferably at least 50%.

Irrespective thereof, it may additionally be preferable that the foam has a retention value R of not more than 90%, especially of not more than 80%, and most preferably of not more than 70%. The retention value R is determined by the method below.

The retention value R describes the maximum amount of water that a foam is able to incorporate into its matrix, without taking into account the water that could be absorbed into the pores. The retention value is determined by punching a 5 cm ×5 cm specimen (stored under standard climatic conditions) from a foam having a thickness of not more than 5 mm and measuring its weight under standard climatic conditions. The specimen is then subjected to free absorption with fluid, especially water, in accordance with DIN EN 13726-1. The amount of fluid absorbed by the pores is squeezed out of the specimen using a roller (weight 5000 g, diameter 10 cm, width 5 cm) by repeatedly placing the sample between fresh cellulose cloths and rolling over it with the roller. This process is repeated until no more fluid absorption is visible in the cellulose cloths. To determine the retention value R, the fluid content Www present in the polyurethane foam after absorption and squeezing out is measured in accordance with DIN EN 14079 and calculated as follows

$$R = {W}_{ww} = \frac{{W}_{gg} - {W}_{tt}}{{W}_{gg}} \cdot 100 \% .$$

where
W_{ww}=weight of the fluid (water) present in the polyurethane foam after absorption and squeezing out,
Wₜₜ=weight of the specimen after drying, and
W_{gg}=weight of the specimen after absorption and squeezing out.

The retention value R of the foam may be at least 20%, preferably at least 30%, especially at least 40%, and most preferably at least 50%.

Irrespective thereof, it may additionally be preferable that the foam has a retention value R of not more than 90%, especially of not more than 80%, and most preferably of not more than 70%.

It is also preferable that the foam is a hydrophilic foam.

In a preferred embodiment, the foam is selected from polyurethane foams and polysiloxane foams. Polyurethane foams are particularly preferred. A polyurethane foam refers to a foam having a polymer matrix that is essentially composed of polyurethane.

Particular preference is given to a hydrophilic, open-cell polyurethane foam. Further preferred hydrophilic polyurethane foams have a density (in the case of foams also referred to as the foam density) of less than 150 kg/m³, especially less than 100 kg/m³, and most preferably 10 to 90 kg/m³.

In preferred embodiments of the employable, especially open-cell, foams, these are polyurethane foams obtainable by the reaction of a curable mixture comprising the components
(i) polyisocyanate,
(ii) isocyanate-reactive compounds, for example polyol, especially polyester polyol,
(iii) catalyst,
(iv) blowing agent, and
(v) optionally additives.

The isocyanates (i) used may be any generally known aliphatic, cycloaliphatic and/or especially aromatic polyisocyanates. Suitable for production of the polyurethanes are, for example, diphenylmethane diisocyanate (MDI), here especially diphenylmethane 4,4′-diisocyanate (4,4′-MDI), mixtures of monomeric diphenylmethane diisocyanates and higher homologs of diphenylmethane diisocyanate (polymeric MDI), tetramethylene diisocyanate (TMDI), hexamethylene diisocyanate (HDI), tolylene diisocyanate (TDI) or mixtures thereof.

Preference is given to MDI, especially 4.4′-MDI and/or HDI. The 4.4′-MDI used with particular preference may contain small amounts of up to about 10% by weight of allophanate- or uretonimine-modified polyisocyanates. It is also possible to use small amounts of polyphenylene polymethylene polyisocyanate (PMDI). The total amount of said PMDI should not exceed 5% by weight of the isocyanate used.

The polyisocyanate component (i) is preferably used in the form of polyisocyanate prepolymers. These polyisocyanate prepolymers are obtainable by reacting polyisocyanates (i) described above, for example at temperatures of 30 to 100° C., preferably at about 80° C., with a substoichiometric amount of the polyols (ii) described below to form the prepolymer. The polyol-polyisocyanate ratio is here chosen such that the NCO content of the prepolymer is 8% to 28% by weight, preferably 14% to 26% by weight, more preferably 17% to 23% by weight.

Polyols such as polyetherols and/or polyesterols are normally used as isocyanate-reactive compounds (ii).

Polyether polyalcohols (referred to in this application as “polyether polyols”) having an OH functionality of 1.9 to 8.0, a hydroxyl value of 50 to 1000 mg KOH/g, and optionally having 10 to 100% of primary hydroxyl groups are possible. Such polyether polyols are known, commercially available, and are based for example on starter compounds that are reacted with alkylene oxides, for example propylene oxide and/or ethylene oxide, under generally known conditions. The content of primary hydroxyl groups can be achieved by finally reacting the polyols with ethylene oxide.

In the production of the foam, preferably of the open-cell foam, preference is given to using polyester polyols in the component (ii). The polyester polyols (ii) used are generally formed by condensation of polyfunctional alcohols, preferably diols, having 2 to 12 carbon atoms, preferably 2 to 6 carbon atoms, with polyfunctional carboxylic acids having 2 to 12 carbon atoms, for example succinic acid, glutaric acid, adipic acid, phthalic acid, isophthalic acid and/or terephthalic acid and mixtures thereof. Examples of suitable di- and polyhydric alcohols are ethanediol, diethylene glycol, butane-1.4-diol, pentane-1.5-diol and/or hexane-1,6-diol and mixtures thereof.

The reaction conditions of the carboxylic acid and alcohol are normally chosen such that the resulting polyester polyols contain no free acid groups. In addition, the resulting polyester polyols generally have a weight-average molecular weight (determined by gel-permeation chromatography) of from 500 to 3000 g/mol, preferably from more than 1000 g/mol to 2500 g/mol. In general, the polyester polyols used have an average theoretical functionality of from 2.0 to 4, preferably from more than 2 to less than 3. In addition, the polyester polyols used generally have an average OH value of from 20 to 200, preferably from 30 to 90.

In a preferred embodiment, the polyester polyols used have a viscosity of from 150 mPa·s to 600 mPa·s, preferably from 200 mPa·s to 550 mPa·s, more preferably from 220 mPa·s to 500 mPa·s, particularly preferably from 250 mPa·s to 450 mPa·s, and especially from 270 mPa·s to 350 mPa·s, measured according to DIN 53 015 at 75° C.

The compounds (ii) may be used in a mixture with chain extenders and/or crosslinkers. Chain extenders are mostly difunctional alcohols having molecular weights of 60 to 499, for example ethylene glycol, propylene glycol, butane-1,4-diol, pentane-1,5-diol, dipropylene glycol and/or tripropylene glycol. Crosslinkers are compounds having molecular weights of 60 to 499 and three or more active H atoms, preferably amines, and more preferably alcohols, for example glycerol, trimethylolpropane and/or pentaerythritol.

In a preferred embodiment, component (ii) contains (or consists of) 0-25% by weight, preferably 1% to 20% by weight, of chain extenders and/or crosslinkers and 75% to 100% by weight, preferably 80% to 99% by weight, of polyol(s), especially polyester polyol(s), based on the total weight of component (ii).

The catalysts (iii) can be customary compounds that accelerate the reaction of component (i) with component (ii). Suitable examples include tertiary amines and/or organic metal compounds, especially tin compounds. For example, the following compounds may be used as catalysts: triethylenediamine, aminoalkyl and/or aminophenyl imidazoles and/or tin(Il) salts of organic carboxylic acids. Catalysts are generally used in an amount of from 0.1% to 5% by weight based on the weight of component (ii).

Generally known chemically or physically acting compounds may be used as blowing agent (iv). As a physically acting blowing agent, preference can be given to the use of water, which forms carbon dioxide through reaction with the isocyanate groups. Examples of physical blowing agents are (cyclo)aliphatic hydrocarbons, preferably ones having 4 to 8, more preferably 4 to 6, and especially 5 carbon atoms, partially halogenated hydrocarbons or ethers, ketones or acetates. The amount of blowing agent used depends on the desired density of the foams. The various blowing agents may be used on their own or in any desired mixtures with one another. Particular preference is given to using just water as blowing agent, generally in an amount of from 0.1% to 5% by weight, especially from 2.5% to 4% by weight, based on the weight of component (ii). Physical blowing agents are preferably used in an amount of <0.5% by weight based on the weight of component (ii).

The reaction is optionally carried out in the presence of auxiliaries and/or additives (v), for example fillers, cell regulators, cell openers, surface-active compounds and/or stabilizers against oxidative, thermal or microbial breakdown or aging.

For the production of polyurethane foams, the components (i) and (ii) are generally reacted in amounts such that the equivalence ratio of NCO groups to the sum total of the reactive hydrogen atoms is 1:0.8 to 1:1.25, preferably 1:0.9 to 1:1.15. A ratio of 1:1 corresponds here to an NCO index of 100. The desired open-cell content of the polyurethane foam is generally ensured by a suitable choice of components (i) to (v) known to those skilled in the art.

According to a further preferred embodiment, the employable, especially open-cell, foams are polyurethane foams obtainable by mechanical beating of aqueous polyurethane dispersions such as Baymedix FD103 and Baymedix AD111 (Covestro). Such foams can advantageously have high mechanical strength and good thermoformability. This makes it possible after beating and curing to finish the foam by thermal processes or thermally cover it with other components, for example a polyurethane film.

According to a further preferred embodiment of the invention, the at least one foam-containing layer comprises a composite of different foams. A particularly advantageous composite includes at least one ply of a polyurethane foam obtainable by mechanical beating of aqueous polyurethane dispersions, and at least one ply of a polyurethane foam obtainable by the reaction of a curable mixture comprising the components
(i) polyisocyanate,
(ii) isocyanate-reactive compounds, for example polyol, especially polyester polyol,
(iii) catalyst,
(iv) blowing agent, and
(v) optionally additives,
   as shown above.

In a wound dressing that comprises such a composite, fluid transport through the wound dressing can be improved. The foam obtainable by mechanical beating normally has larger pores and is arranged toward the wound side. The foam obtainable by chemical reaction (as shown above) normally has finer pores and is arranged over the larger-pored foam on the side facing away from the wound. This arrangement is able to generate a capillary effect that can additionally facilitate the transport of fluid from the wound.

Any wound-contact layer known from the prior art is in principle suitable as a wound-contact layer (c), provided it both guarantees the passage of wound exudate and the material has no tendency to fuse or stick to the wound tissue.

Examples of suitable wound-contact layers are described in German patent applications DE 10 2008 062 472, DE 10 2008 031 183, and DE 10 2008 031 182.

In a preferred embodiment, the wound-contact layer comprises a hydrophobic material. The wound-contact layer particularly preferably comprises a silicone, especially an adhesive silicone.

The wound-contact layer may for example be a silicone mesh, a silicone foil or a silicone film. Alternatively, the wound-contact layer may be a mesh, foil or film made of a polymer material that has been siliconized, i.e. the polymer material has been treated with silicone that is largely bonded to the surface of the polymer material. As another alternative, silicone may already be added during formation of the polymer or even be a participant in the production of the polymer material for the wound-contact layer, with the result that a silicone-containing polymer material is formed.

In a preferred embodiment, the wound-contact layer may be perforated, i.e. the wound-contact layer may have perforations. A perforation may be regarded as an opening (hole) that passes through the wound-contact layer. Perforations may be obtained using any suitable perforation device such as nails, needles or a punch. Alternatively, perforation may also be executed for example by punching, diecutting (scoring), ultrasonic cutting or ultrasonic punching.

The perforation holes may be any shape. For example, the perforation holes may be rectangular, square, circular, elliptical, triangular, pentagonal, hexagonal, octagonal or diamond-shaped. Preferably, the perforation holes are square, circular or elliptical, especially circular.

The perforation holes preferably have a size of between 1.75 mm² and 12.5 mm^{2,} more preferably between 2.0 mm² and 8.5 mm², especially between 2.25 mm² and 5.0 mm².

When the perforation is circular, a perforation hole of between 1.75 mm² and 12.5 mm² in size corresponds to a perforation diameter of from about 1.5 mm to about 3.9 mm. Similarly, again assuming a circular perforation, a perforation hole of between 2.25 mm² and 5.0 mm² in size corresponds to a perforation diameter of from about 1.7 mm to about 2.5 mm.

Preferably, the sum total of the areas of the perforation holes is between 10% and 50%, more preferably between 12% and 45%, especially between 15% and 30%, of the total area of the wound-contact layer.

The sum total of the areas of the perforation holes is preferably about 15%, about 16%, about 17%, about 18%, about 19%, about 20%, about 21%, about 22%, about 23%, about 24%, about 25%, about 26%, about 27%, about 28%, about 29%, or about 30%, preferably about 25%.

According to a preferred embodiment, the wound-contact layer has perforations having a perforation pattern such that, in the wound-contact layer, the average pull-off force in the machine direction and average pull-off force in the transverse direction (thereto) do not differ from one another by more than 5%, preferably not more than 4%, especially not more than 3%.

Alternatively preferably, the wound-contact layer has perforations having a perforation pattern such that, in the wound-contact layer, the variation between the highest and lowest values of the pull-off force curve is not more than 25%, preferably not more than 15%, of the average pull-off force.

The pull-off force is determined according to a method based on FINAT No. 1, with the pull-off force determined over a width of 25 mm.

In a preferred embodiment, the perforations in the wound-contact layer are arranged the same distance apart such that they form a square oriented in line with the machine and transverse directions.

In an alternatively preferred embodiment, the perforations in the wound-contact layer are arranged the same distance apart such that they form a square oriented at an angle of 45° to the machine and transverse directions.

Alternatively, the perforations in the wound-contact layer are arranged the same distance apart such that they form an equilateral triangle wherein the holes are oriented at an angle of between 10° and 20° to the machine and transverse directions.

In a preferred embodiment, the wound-contact layer consists of a PU membrane coated on both sides, wherein the wound-facing side is charged with a silicone gel, whereas the side facing the wound foam is coated with an acrylate adhesive (Acrysil 150 from Advanced Silicone Coating S.A.S.).

The wound dressing of the invention includes at least one textile-based fabric (d), which contains at least one sensor. In an alternatively preferred embodiment, the wound dressing of the invention includes two separate textile-based fabrics, each containing at least one sensor. In an alternatively preferred embodiment, the wound dressing of the invention includes three separate textile-based fabrics, each containing at least one sensor.

The at least one textile-based fabric containing at least one sensor may take the form of a textile-based fabric as described above, for example a woven fabric, knitted fabric, nonwoven fabric, braid, bobbinet, tulle, net, or felt.

In a preferred embodiment, the at least one textile-based fabric containing at least one sensor is a knitted fabric, preferably a warp-knit fabric.

The at least one sensor contained in the at least one textile-based fabric may be a sensor known to those skilled in the art.

For example, the sensor may be a sensor based on a thermoelectric, resistive, capacitive, inductive, optical, acoustic, magnetic, chemical or other operating principle. The sensor may for example be an integrated circuit (“microchip”).

In a preferred embodiment, the at least one sensor contained in the at least one textile fabric may be designed as an electrically conductive thread.

A thread is a flexible structure that has a dominant one-dimensional extent and a uniformity in the longitudinal direction. A thread also includes yarns and twists.

The electrically conductive thread is able to conduct electrical current. The conductive thread may preferably be designed as a conductive wire. This wire may have a diameter of from 10 μm to 250 μm, preferably from 20 μm to 150 μm, more preferably from 30 μm to 100 μm, especially from about 50 μm.

An electrically conductive thread is a thread that includes an electrically conductive material, in particular is coated therewith, or consists of a conductive material. For example, a chosen thread may have an electrically conductive coating through which electrical conductivity of the thread is brought about.

Conductive material is understood as meaning a material that is able to conduct electrical current. Examples include metals such as silver, copper, aluminum, zinc, and iron. Alternatively, alloys of metals such as brass may be used.

Also suitable as materials for the conductive thread(s) are conductive polymers and semiconductors such as silicon as an elemental semiconductor, GaP, ZnO, CdS, GaTe or SiC as compound semiconductors, and tetracene, acridone, indanthrone or Alq3 as organic semiconductors.

Graphite is also suitable as a material for the conductive thread(s).

A thread considered to be electrically conductive has an electrical resistance here of not more than 2000 ohm/m, preferably of not more than 1000 ohm/m, more preferably of not more than 500 ohm/m. This means that a thread of 1 m in length has an electrical resistance, measured in the customary manner, of respectively not more than 2000 ohms, not more than 1000 ohms, or not more than 500 ohms. To check whether or not a thread is electrically conductive, conditioned threads are used under standard laboratory conditions, i.e. threads that are dry and have been conditioned for at least 24 hours at a temperature of 20° C. and a relative humidity of 65%.

The conductive thread/wire is preferably insulated against the environment, both to provide accurate measurement data and to prevent possible adverse effects on wound healing caused by the flow of electricity. The insulating materials known to those skilled in the art can be used for this purpose.

In a preferred embodiment, the at least one conductive thread is an enameled copper wire, especially an enameled copper wire insulated with a polyurethane.

In an alternatively preferred embodiment, the at least one sensor contained in the at least one textile-based fabric may be designed as a conductive filament.

Filaments refers to fibers of practically unlimited length (“continuous fibers”). A filament may be prepared by wet, dry or melt spinning. A filament may include fibers from raw materials of natural origin, for example a viscose filament. Alternatively, a filament may include fibers of synthetic origin. For example, a filament produced by a melt spinning process may include fibers composed of thermoplastics such as polyester. There are also filaments that can include fibers of both natural and synthetic origin. A conductive filament additionally contains a conductive material, preferably a metal, a polymer or copolymer. The conductive material may be present as a coating on a non-conductive base material.

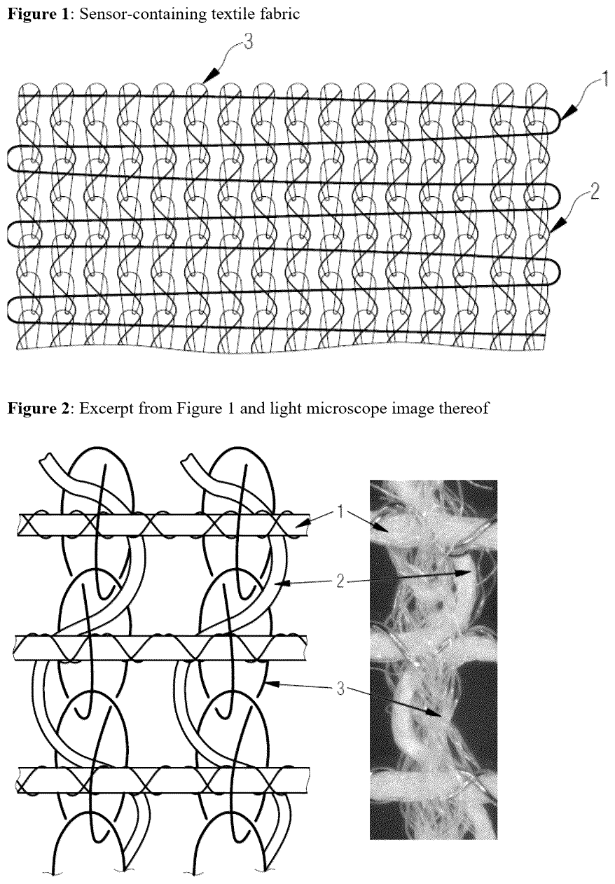

In a preferred embodiment, the at least one sensor contained in the at least one textile fabric is arranged in an ordered structure in the textile-based fabric. An ordered structure is understood as meaning a repeating structure in which the individual thread systems have a defined dimensional orientation/thread direction in the textile fabric. A textile fabric containing a sensor according to the invention is shown in FIG. 1, wherein the textile fabric containing a sensor comprises a sensor yarn (1), a viscose yarn (2), and a textured polyamide yarn (3). FIG. 2 shows an excerpt from FIG. 1 and a light microscope image thereof.

This makes it possible, depending on the operating principle of the sensor, to generate or else to advantageously reduce an interaction of magnetic fields generated by the flow of current through the conductive thread(s) and/or the conductive filament(s), thereby making it possible to achieve a higher sensor accuracy and register a less interference-prone, analog sensor signal.

In a preferred embodiment, the at least one textile-based fabric containing a sensor is a warp-knit fabric wherein preferably at least one thread and/or filament of the thread systems has conductive properties and can be employed as a sensor. The thread with sensory function may be a PES f48 yarn produced by TWD Fibres GmbH, around which are wound two enameled copper wires from Superior ESSEX, wherein the at least one sensor is integrated by means of weft insertion technology. This can be done for example by means of a Comez 609 crochet galloon machine. In weft insertion technology, the at least one conductive thread (sensor thread) can be integrated into the textile-based fabric in the form of a full weft or in the form of a partial weft. The at least one sensor integrated into the at least one textile-based fabric as a full weft or partial weft is preferably able to cover a relatively large area and thus, depending on the applied sensor principle, result in greater accuracy of measurement.

The ends of the conductive thread and/or of the conductive filament of the at least one sensor contained in at least one textile-based fabric are each finally led out under the cover layer and are there provided with a connection such that a device, especially a device that includes a microprocessor/microcontroller, can be connected to read off the parameters to be determined.

In a preferred embodiment, the at least one sensor contained in at least one textile-based fabric is able to measure the content of fluid, the temperature or the pH. In other words, the at least one sensor contained in at least one textile-based fabric may preferably be a fluid sensor, a temperature sensor or a pH sensor.

For example, a fluid-measuring sensor may be used to measure the moisture in the wound. It is then possible, for example after a measurement has confirmed an amount of moisture that is advantageous for wound healing, to forgo a dressing change.

In addition to the moisture in the wound, the amount of fluid absorbed into the foam can however also be determined by means of the sensor described above. This is very important, because the foam has only a limited absorption capacity. In order to maintain a climate favorable to wound healing, the dressing should be changed before this absorption capacity (“foam filling level”) has been reached.

It is also possible for the temperature of the wound to be measured by means of a temperature-measuring sensor. The temperature obtained allows conclusions to be drawn about the condition of the wound or the presence of inflammation, making it possible to then decide whether a dressing change is necessary.

With the aid of a sensor that determines the pH of the wound, it is possible to establish for example whether an environment favoring the growth of bacteria and biofilms is present and to act accordingly, for example by changing the dressing and administering appropriate active substances.

The textile-based fabric may contain at least one sensor. In a preferred embodiment, the textile-based fabric contains one sensor. The sensor may for example be a fluid sensor as a first sensor, a temperature sensor and/or pH sensor.

In a preferred embodiment, the textile-based fabric contains two sensors. In a preferred embodiment, the textile-based fabric may contain two identical sensors, for example two fluid sensors or two temperature sensors. In an alternatively preferred embodiment, the textile-based fabric may contain two different sensors, for example a fluid sensor as a first sensor and a temperature sensor or pH sensor as a second sensor.

In a preferred embodiment, the textile-based fabric contains three sensors. In a preferred embodiment, the textile-based fabric may contain three identical sensors, for example three fluid sensors or three temperature sensors. In an alternatively preferred embodiment, the textile-based fabric may contain different sensors, for example two fluid sensors and one temperature sensor. Alternatively preferably, the textile-based fabric may contain a fluid sensor as a first sensor, a temperature sensor as a second sensor, and a pH sensor as a third sensor.

This allows the temperature, the presence of fluid and/or the pH to be separately measured by the at least one sensor contained in the at least one textile-based fabric.

In a preferred embodiment of the invention, the wound dressing includes two foam-containing layers. What is described above applies to the two foam-containing layers. The two foam-containing layers preferably comprise the same foam. Alternatively preferably, the two foam-containing layers comprise different foams that are based for example on different polymer matrices.

In a preferred embodiment, the two foam-containing layers may contain foams of different pore size, with the foam having the larger pore size facing the wound. Such an arrangement has the advantage of making it possible to exploit the resulting capillary effects.

In a preferred embodiment of the invention, the at least one sensor contained in at least one textile-based fabric is arranged between the cover layer and foam-containing layer, in the foam-containing layer, between two foam-containing layers, between the foam-containing layer and the wound-contact layer and/or in the wound-contact layer.

In one embodiment, the at least one sensor contained in at least one textile-based fabric is arranged between the cover layer and foam-containing layer or in the foam-containing layer, preferably in the vicinity of the cover layer. The sensor is preferably a fluid sensor, by means of which the fluid, more particularly the aqueous, content of the foam-containing layer can be measured and the (remaining) absorption capacity of the foam determined or estimated therefrom. Based on the (residual) absorption capacity of the foam, it is possible to determine whether a dressing change is necessary or can be dispensed with.

In one embodiment, the at least one sensor contained in at least one textile-based fabric is arranged between two foam-containing layers. What is described above applies to the two foam-containing layers. Preferably, at least one of the sensors is a fluid sensor, by means of which preferably the aqueous/fluid content of the foam-containing layer facing the wound can be measured and the (remaining) absorption capacity of the foam determined therefrom. Based on the (residual) absorption capacity of the foam, it is possible to determine whether a dressing change is necessary or can be dispensed with.

In one embodiment, the at least one sensor contained in at least one textile-based fabric is arranged between the foam-containing layer and the wound-contact layer. Preferably, the at least one sensor is a fluid sensor, by means of which the moisture in the wound can be measured and the condition of the wound and possible measures such as the need for a dressing change inferred therefrom.

In one embodiment, the at least one sensor contained in at least one textile-based fabric is arranged between the foam-containing layer and the wound-contact layer. Preferably, the at least one sensor is a temperature sensor, by means of which the temperature of the wound can be measured and the condition of the wound and possible measures such as a dressing change inferred therefrom.

In one embodiment, the at least one sensor contained in at least one textile-based fabric is arranged between the foam-containing layer and the wound-contact layer. Preferably, the at least one sensor is a pH sensor, by means of which the pH of the wound can be measured and the condition of the wound and possible measures such as a dressing change inferred therefrom.

In one embodiment, the at least one sensor contained in at least one textile-based fabric is arranged between two foam-containing layers. Preferably, the at least one sensor is a pH sensor, by means of which the pH of the fluid secreted from the wound (wound exudate) can be measured and the condition of the wound and possible measures such as a dressing change or administration of active substances inferred therefrom.

A further aspect of the invention is a process for producing a wound dressing of the invention, comprising the steps of
(i) providing (a) a cover layer, (b) at least one foam-containing layer, (c) a wound-contact layer, and (d) at least one textile-based fabric containing at least one sensor, and
(ii) arranging (a) a cover layer, (b) at least one foam-containing layer, (c) a wound-contact layer, and (d) at least one textile-based fabric containing at least one sensor, wherein the at least one textile-based fabric containing at least one sensor is arranged between (a) the cover layer and (b) the at least one foam-containing layer, between two foam-containing layers (b1) and (b2) and/or (b) the at least one foam-containing layer and (c) the wound-contact layer, or in (c) the wound-contact layer.

In step (i), (a) a cover layer, (b) at least one foam-containing layer, (c) a wound-contact layer and (d) at least one textile-based fabric containing at least one sensor are provided. The above applies to (a) the cover layer, (b) the at least one foam-containing layer, (c) the wound-contact layer, and (d) the at least one textile-based fabric containing at least one sensor.

In step (ii), (a) a cover layer, (b) at least one foam-containing layer, (c) a wound-contact layer and (d) at least one textile-based fabric containing at least one sensor are arranged on top of one another, wherein the at least one textile-based fabric containing at least one sensor is arranged between (a) the cover layer and (b) the at least one foam-containing layer, between two foam-containing layers (b1) and (b2) and/or (b) the at least one foam-containing layer and (c) the wound-contact layer, or in (c) the wound-contact layer.

The (d) at least one textile-based fabric containing at least one sensor can preferably be arranged between (a) the cover layer and (b) the foam-containing layer, in (b) the foam-containing layer, between two foam-containing layers (b1) and (b2) and/or between (b) the foam-containing layer and (c) the wound-contact layer.

In a preferred embodiment, the wound dressing of the invention comprises
(a) a film as a cover layer,
(b) an absorbent foam as a foam-containing layer,
(c) a silicone as a wound-contact layer, and
(d) a textile-based fabric containing a sensor.

More preferably, the wound dressing of the invention comprises
(a) a polyurethane film as a cover layer,
(b) a polyurethane foam as a foam-containing layer,
(c) a silicone layer, preferably a silicone mesh, as a wound-contact layer, and
(d) a warp-knit fabric containing a sensor.

In a particularly preferred embodiment, the wound dressing of the invention comprises
(a) a polyurethane film as a cover layer,
(b) a polyurethane foam as two foam-containing layers,
(c) a silicone layer, preferably a silicone mesh, as a wound-contact layer, and
(d) a warp-knit fabric containing a sensor,
   wherein the warp-knit fabric containing the sensor is arranged between two foam-containing layers made of polyurethane foam and wherein the two foam-containing layers and the warp-knit fabric containing the sensor is arranged between the silicone layer, preferably a silicone mesh, and the cover layer, preferably a polyurethane film.

A further aspect is the use of a wound dressing of the invention for covering a wound space for a period of from one to 14 days. Preferably, the wound dressing may be used to cover the wound for a period of from one to twelve days, one to ten days, one to eight days, one to six days, one to five days, one to four days, one to three days or one to two days.

Alternatively preferably, the wound dressing may be used to cover the wound for a period of from two to 14 days, two to twelve days, two to ten days, two to eight days, two to six days, two to five days, two to four days, or two to three days.

Alternatively preferably, the wound dressing may be used to cover the wound for a period of from three to 14 days, three to twelve days, three to ten days, three to eight days, three to six days, three to five days or three to four days.

This applies in particular when the sensors can be used to determine whether both the wound and the dressing are in a condition that precludes the need for a dressing change. This makes it possible to avoid unnecessary dressing changes.

A further aspect of the invention is a conductive thread and/or conductive filament contained in a wound dressing of the invention for use in the treatment of chronic wounds, where the treatment involves the determination of wound parameters. Again, it can be stated that the determination of wound parameters makes it possible to avoid unnecessary dressing changes and resulting trauma.

A further aspect of the invention is a conductive thread and/or conductive filament contained in a wound dressing of the invention for use in diagnosing the condition of chronic wounds, where this diagnosing involves the determination of wound parameters. It can be stated that the determination of wound parameters makes it possible to avoid unnecessary dressing changes and resulting trauma.

A further aspect of the invention is the use of a conductive thread contained in a textile-based fabric in the diagnosing of wounds, especially of chronic wounds. The use of a conductive thread contained in a textile-based fabric makes it possible to determine wound parameters. Based on these determined wound parameters, it is possible to avoid unnecessary dressing changes and the resulting trauma or to arrange appropriate treatment measures such as dressing changes or administration of active substances.

A further aspect of the invention is the use of the wound dressing of the invention in the diagnosing of wounds, especially of chronic wounds. The use of a conductive thread contained in a textile-based fabric makes it possible to determine wound parameters. Based on these determined wound parameters, it is possible to avoid unnecessary dressing changes and the resulting trauma or to arrange appropriate treatment measures such as dressing changes or administration of active substances.

The invention is to be elucidated by the examples that follow.

### EXAMPLES

### Example 1: Constituents of the Wound Dressing of the Invention

1(a) The cover layer is for example a polyurethane film having a thickness of 25 μm such as Platilon U073 from Covestro.

1(b) The foam-containing layer is for example a polyurethane foam having a thickness of about 3 mm obtainable for example by the reaction of Baymedix FD103 and AD111.

1(c) The wound-contact layer is for example a perforated multilayer silicone layer (Acrysil 150 from Advanced Silicone Coating S.A.S.) or a silicone web having a thickness of 150 μm.

1(d) The at least one textile-based fabric containing a sensor can be produced for example as a warp-knit fabric on a Comez 609 crochet galloon machine, it being possible for the sensor to be integrated by means of weft insertion technology. For this, a conductive thread as sensor and a yarn for production of the warp-knit fabric can be used with the following parameters given by way of example: The conductive thread is an enameled copper wire from Superior Essex (USA) having a diameter of 50 μm, which has a polyurethane insulating layer with a thickness of 0.02 μm. The yarn is a polyester yarn (TWD PES f48) from TWD Fibres GmbH (Germany).

The sensor yarn was produced by the DITF (German Institute for Textile Research, Denkendorf) research institute. A textile-based fabric of this kind containing a sensor is shown in FIG. 1. The sensor yarn (1) shown in FIG. 1 was produced by the DITF (German Institute for Textile Research, Denkendorf) research institute. FIG. 2 shows an excerpt from FIG. 1 and a light microscope image thereof.

### Example 2: Structure of a Wound Dressing of the Invention

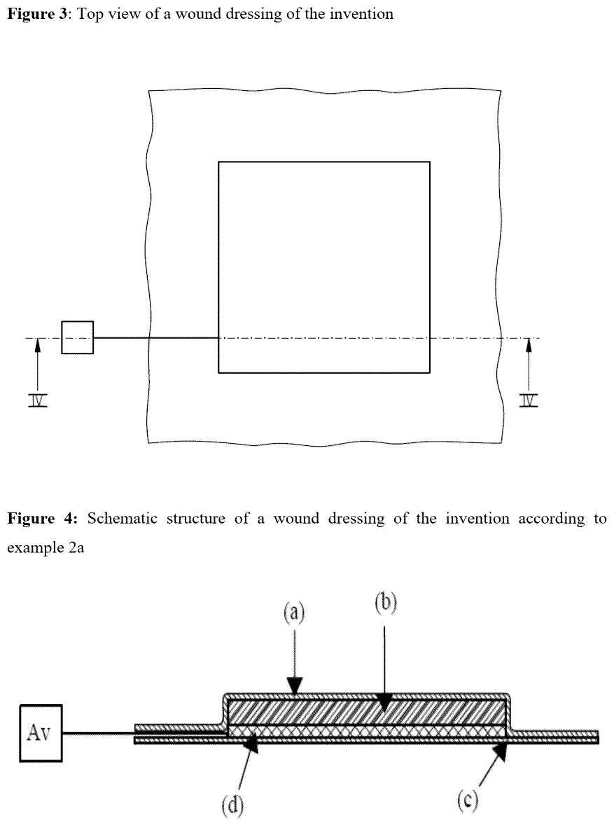

FIG. 3 shows the top view of a wound dressing of the invention, in which the island form of the wound dressing can be seen.

Example 2a: FIG. 4 shows the cross-sectional view of a schematic structure of a wound dressing of the invention along the IV-IV line of FIG. 3. The figure shows how the textile-based fabric containing a sensor (“sensor knit”) (d) is arranged on a silicone mesh serving as a wound-contact layer (c). Positioned on the sensor knit is a polyurethane foam as a foam-containing layer (b), with a polyurethane film as cover layer (a) arranged thereabove. Av denotes a reading device.

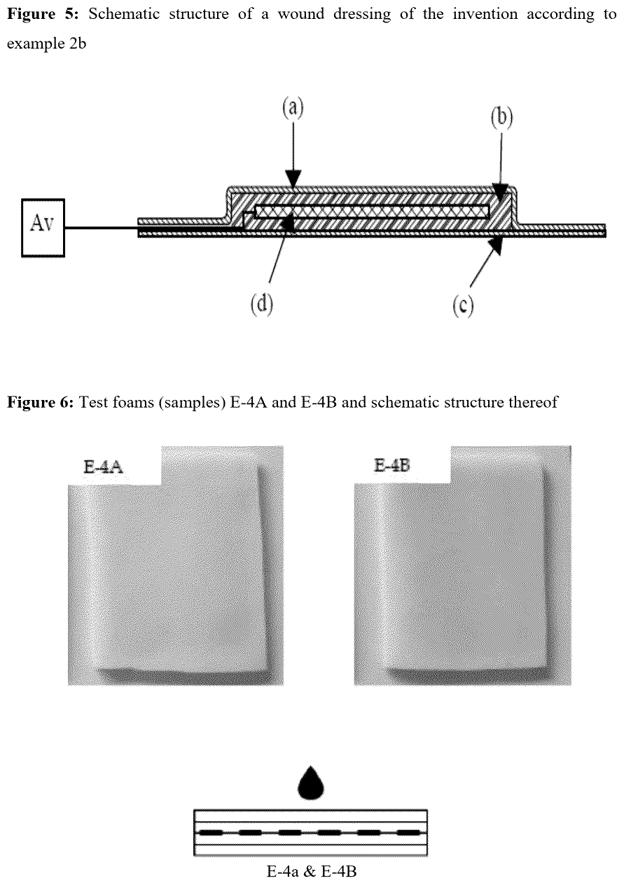

Example 2b: FIG. 5 shows the cross-sectional view of a schematic structure of a wound dressing of the invention along the IV-IV line of FIG. 3. The figure shows how a polyurethane foam as a foam-containing layer (b) is arranged on a silicone mesh serving as a wound-contact layer (c), the foam-containing layer (b) including a sensor-containing textile-based fabric. Arranged above the foam-containing layer (b) is a polyurethane film as a cover layer (a). Av denotes a reading device.

### Example 3: Determination of the Absorption Capacity of a Foam by Means of a Sensor Contained in a Textile-Based Fabric

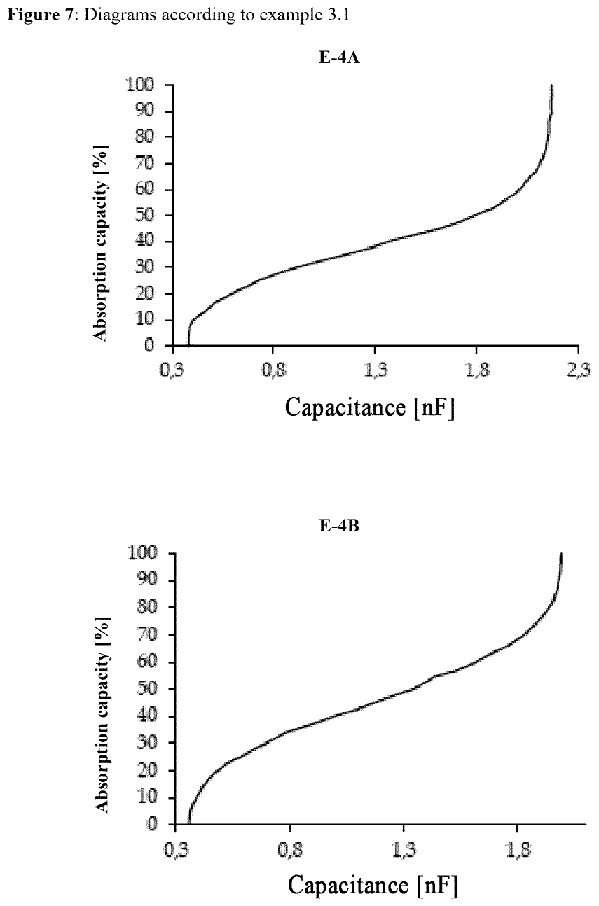

3.1 For determination of the absorption capacity (“filling level”) of two foams having a thickness of 3 mm, a sensor contained in a textile-based fabric is integrated into each of the foams. FIG. 6 shows the two test foams E-4A and E-4B (top) and a schematic structure thereof (bottom).

The capacity is then determined at maximum absorption. This is done by testing the free absorption capacity of the sample in accordance with DIN EN 13726-1. In a departure from the standard, the associated electrical capacitance value is included in the weight value after 30 minutes of free absorption in a test solution prescribed in the DIN that was determined here. This first test gives the maximum absorption capacity [g], which corresponds to an absorption capacity (filling level) of 100% and the associated electrical capacitance value. In the test that follows, the absorption curve (filling level trajectory) is determined. This is done by discontinuously loading the sample over a certain time interval (in this case approx. 45 minutes) with the same amount of a test fluid at exact intervals of one minute, until the previously determined absorption capacity (=100% filling level) is reached. The weight and electrical capacitance value each minute are dated. From the weight value, the filling level is determined and the corresponding capacity value assigned. The corresponding diagrams are shown in FIG. 7. As can be seen from the diagrams, from about 70% of the absorption capacity in the case of for example sample E-4A (upper figure) and from about 80% for sample E-4B (lower figure) the electrical capacitance no longer increases strongly but stagnates. From this it can be inferred that the absorption capacity of the foam is (almost) exhausted and the need for a change is imminent.

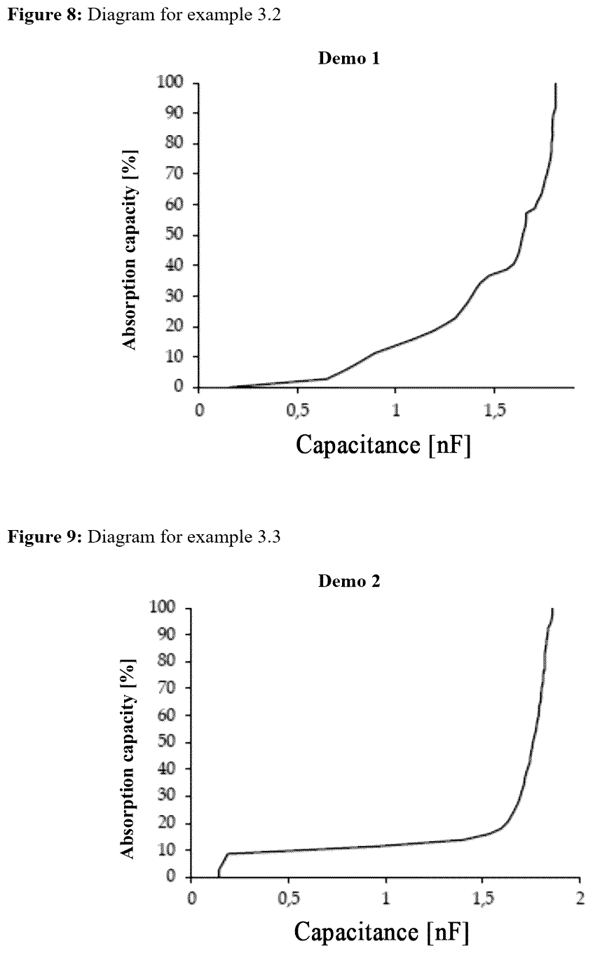

3.2 For determination of the absorption capacity (“filling level”) of the foam, a sensor contained in a textile-based fabric is arranged between a silicone layer (which may correspond to the wound-contact layer) and a foam. The absorption capacity (filling level) of the foam (in %) and electrical capacitance of the sensor (in nF) are then determined in the same way as described above. The corresponding diagram is shown in FIG. 8. As can be seen from the diagram, the capacitance of the sensor comes up against a threshold value from which it can be inferred that the absorption capacity of the foam is (almost) exhausted and the need for a change is imminent.

3.3 For determination of the absorption capacity (“filling level”) of the foam, a sensor contained in a textile-based fabric is arranged between a film cover (which may correspond to the cover layer) and a foam. The filling level (in %) and capacitance of the sensor (in nF) are then determined in the same way as described above. The corresponding diagram is shown in FIG. 9. As can be seen from the diagram, above a certain threshold value the capacitance increases further only slowly and approaches a threshold value from which it can be inferred that the absorption capacity of the foam is (almost) exhausted and the need for a change is imminent.

1. A wound dressing comprising:
(a) cover layer,
(b) at least one foam-containing layer,
(c) wound-contact layer, and
(d) at least one textile-based fabric containing at least one sensor.

2. The wound dressing as claimed in claim 1, wherein the cover layer is a film or a textile-based fabric.

3. The wound dressing as claimed in claim 1 or 2, wherein the foam is an absorbent foam.

4. The wound dressing as claimed in any of the preceding claims, wherein the foam is a polyurethane foam, especially a hydrophilic polyurethane foam.

5. The wound dressing as claimed in any of the preceding claims, wherein the wound-contact layer comprises a silicone.

6. The wound dressing as claimed in any of the preceding claims, wherein the at least one sensor contained in the at least one textile-based fabric is a conductive thread and/or a conductive filament.

7. The wound dressing as claimed in claim 6, wherein the conductive thread includes an insulated wire made of a conductive material, and/or the conductive filament comprises a conductive polymer.

8. The wound dressing as claimed in any of the preceding claims, wherein the at least one sensor contained in the at least one textile-based fabric is arranged in an ordered structure in the textile-based fabric.

9. The wound dressing as claimed in any of the preceding claims, wherein the at least one sensor contained in the at least one textile-based fabric separately measures the presence of fluid, the temperature and/or the pH.

10. The wound dressing as claimed in any of the preceding claims, wherein the wound dressing includes two foam-containing layers.

11. The wound dressing as claimed in any of the preceding claims, wherein the at least one sensor contained in the at least one textile-based fabric is arranged between the cover layer and the foam-containing layer, in the foam-containing layer, between two foam-containing layers and/or between the foam-containing layer and the wound-contact layer.

12. The wound dressing as claimed in any of the preceding claims, wherein the wound-contact layer includes at least one sensor contained in the at least one textile-based fabric.

13. The wound dressing as claimed in any of the preceding claims, comprising
(a) a film as a cover layer,
(b) an absorbent foam as a foam-containing layer,
(c) a silicone as a wound-contact layer, and
(d) a textile-based fabric containing a sensor.

14. The wound dressing as claimed in any of the preceding claims, comprising
(a) a polyurethane film as a cover layer,
(b) a polyurethane foam as a foam-containing layer,
(c) a silicone layer, preferably a silicone mesh, as a wound-contact layer, and
(d) a warp-knit fabric containing a sensor.

15. The wound dressing as claimed in any of the preceding claims, comprising
(a) a polyurethane film as a cover layer,
(b) a polyurethane foam as a foam-containing layer,
(c) a silicone layer, preferably a silicone mesh, as a wound-contact layer, and
(d) a warp-knit fabric containing a sensor,
wherein the warp-knit fabric containing a sensor is arranged between the foam-containing layer and the wound-contact layer.
